(19) United States
(12) Patent Application Publication
(10) Pub. No.: US 2025/0205033 A1
(43) Pub. Date: Jun. 26, 2025

(54) Method for inducing an improvement in the mechanical strength of an implantable device comprising one or more yarns of PLA at the end of a determined hydrolysis, and implantable device which can be obtained by said method

(71) Applicant: COUSIN BIOTECH, Wervicq Sud (FR)

(72) Inventors: Gilles SOLECKI, WERVICQ SUD (FR); Safa OUERGHEMMI, WERVICQ SUD (FR)

(21) Appl. No.: 18/987,116

(22) Filed: Dec. 19, 2024

(30) Foreign Application Priority Data
Dec. 21, 2023 (FR) 2314790

(51) Int. Cl. A61F 2/00

(52) U.S. Cl. CPC A61F 2/0063; A61F 2002/0068; A61F 2210/0004; A61F 2240/001

(57) ABSTRACT

The present disclosure relates to a method for inducing an improvement in the mechanical strength (N/cm) of an implantable device after a determined period of hydrolysis. The method includes a step of providing an implantable device that has one or more yarns comprising at least one lactic acid (co)polymer; a step of treating the implantable device with supercritical carbon dioxide (CO₂); and a step of obtaining an implantable device having a mechanical strength greater than or equal to 10 N/cm, preferably greater than or equal to 25 N/cm, after 20 weeks of hydrolysis, in particular in an aqueous medium. The present disclosure also relates to an implantable device that can be obtained by the method, and the use of supercritical (CO₂) for inducing an improvement in the mechanical strength of an implantable device comprising one or more yarns comprising a lactic acid (co)polymer.

### TECHNICAL FIELD

The present disclosure relates to the technical field of implantable devices that are at least partially resorbable and at least partially textile, in particular for the treatment of hernias, having an improved mechanical strength at the end of a determined prolonged period of hydrolysis.

The present disclosure also relates to the technical field of methods for improving the mechanical strength at the end of a prolonged period of hydrolysis of implantable devices that are at least partially textile and at least partially resorbable, in particular for the treatment of abdominal hernias.

### BACKGROUND

It is known to use at least partially textile and at least partially resorbable or non-resorbable implantable devices for the treatment of abdominal hernias.

These implantable devices generally comprise an implantable textile panel, that is at least partially resorbable or non-resorbable, substantially flat and/or having an anatomical shape in three dimensions in order to fill a parietal defect. The textile panel is generally a knitted fabric or a fabric, for example a knitted fabric of monofilamentary yarns contributing sufficient mechanical strength, made of polypropylene or polyethylene for example when non resorbable, or made of P4HB for example when resorbable. When the reinforcing textile panel is non-resorbable, it is intended to remain implanted in the patient in treatment of the hernia. When the reinforcing textile panel is resorbable, it is intended to be hydrolysed by the natural implantation medium of the patient which is an aqueous medium and therefore has disappeared at the end of a determined period. The choice of a resorbable or non-resorbable implantable device depends, in particular, on the type of patient, in particular whether the patient has risks such as obesity, diabetes, smoking, or even a combination of these different risks.

It has been observed that patients presenting with one or more of the risk factors listed above are more prone to the risk of abdominal hernia recurrence, and above all to infections at the implantable device. These infections, when they cannot be treated by a drug treatment, require a surgical operation in order to remove the implantable device. These infections generally arise after a period of several months. Surgical practice in the field of ventral hernia treatment thus tends to no longer use implantable devices made of polypropylene or polyester, in other words non-resorbable materials, in order to treat patients with infection risk factors because these patients will be required to undergo corrective surgery consisting of removing the implantable device. In order to overcome this disadvantage, patients with infection risk factors are treated with resorbable implantable devices. Thus, in the treatment of abdominal hernias for these infection-risk patients, the implantable device is known marketed under the tradename PHASIX® with resorbable P4HB yarns, with complete resorption after 18 months. Thus, in the case of infections after several months of implantation, it is not necessary to remove the implanted device.

The commercially available resorbable implantable devices make it possible to relatively correctly treat the infection without having to explant the implanted device (since it disappears totally over time) but the probability of recurrence of the hernia is greater.

For infection-risk patients, the majority of recurrences appear before the end of the first year after implantation, in other words up to 12 months. These recurrences which occur during the first year appear at the periphery of the implanted device, in other words on anatomical areas which are not covered by the implanted device. After the first year, the recurrences appear on the anatomical areas covered by the resorbable implanted device, which breaks due to a loss of strength due to degradation by hydrolysis.

It is thus sought to improve the mechanical strength of resorbable implanted devices during hydrolysis, several months after start of the hydrolysis, in order to avoid a recurrence of the hernia.

The publication entitled “In Vitro Degradation of Poly(L-lactic acid) fibers produced by melt spinning”; A. Pegoretti, L. Fambri, C. Migliaresi, Department of Materials Engineering, University of Trento, via Mesiano 77, 38050 Trento, Italy, highlights the mechanical properties of fibres with the smallest diameters (approximately 72 μm) degrading quicker than those of fibres for which the diameter is higher (approximately 120 μm) under the effect of the hydrolysis of the PLLA. This study revealed that the rate of degradation depended on a number of factors, including the type of polymer, the manufacturing conditions of the PLLA (by the molten or solvented pathway) and the degradation medium. In particular, the lower the surface to volume ratio and the higher the initial molar mass, the more the rate of degradation of the molar mass and the mechanical properties are slowed down. It is observed that after 16 weeks (4 months), the breaking strength (MPa), shown in FIG. 6, jumps from 900 MPa to approximately 225 MPa for a monofilament of diameter 120 microns, i.e. a loss of mechanical strength of order 75% for a hydrolysis period of 4 months. For fibres having a diameter of 72 microns, the breaking strength drops from 975 MPa to 110 MPa after 4 months of hydrolysis (i.e. a loss of mechanical strength of 89%, cf. FIG. 7b). The 120 μm fibres retain a breaking strength greater than that of the 72 μm fibres, but the degradation in breaking strength is greater than or equal to 75% in both cases after a hydrolysis period of 4 months in a buffered solution at pH 7.4 and at a temperature of 37° C.

A person skilled in the art is thus led to increase the diameter of the filaments of the implantable devices in order to slow the loss of mechanical strength; Nevertheless, too large a loss of mechanical strength (70%) is observed at the end of 4 months. However, a mechanical strength is sought, which is not too low at the end of 4 months, or even more significantly at the end of 5 months, and at the end of 8 months, since the risks of infection and recurrence are greater around 12 months. Moreover, it is not possible to use filaments having too large a diameter, because the implantable device must remain flexible, in particular sufficiently flexible so that it can be rolled up on itself and deposited in an insertion trocar of several millimetres diameter, and then unrolled at the implantation site.

It has thus been proposed to add a resorbable coating on the filaments which are themselves resorbable, in order to slow the hydrolysis of the yarns and delay the loss of mechanical strength. Nevertheless, this provision requires a step of coating the yarns, and a tendency to stiffen the yarns, which is to the detriment of patient comfort, and reduces the flexibility of the implantable device.

An object of the present disclosure is therefore a method for inducing an improvement in the mechanical resistance to hydrolysis, in particular in an aqueous medium, of an implantable device comprising one or more monofilamentary yarns of lactic acid (co)polymers, and this without losing the flexibility of the implantable device in order that it can be inserted in a trocar.

Another object of the present disclosure is a totally or partially resorbable implantable device, comprising one or more yarns of lactic acid (co)polymer, and having an improved mechanical resistance to hydrolysis, in particular in aqueous medium, in particular at 5 months and/or at 8 months.

### DESCRIPTION OF THE DRAWINGS

The present disclosure will be better understand on reading the following embodiments, cited by way of non-limiting examples, and illustrated by the figures, in which:

FIG. 1 schematically shows the various states (solid, liquid, gas, supercritical) of carbon dioxide as a function of the pressure (bar) as ordinate and temperature (° C.) as abscissa;

FIG. 2 is a table showing the values of mechanical strength (ball bursting value) for an example according to this disclosure (EX1) and two comparative examples (EXC1, EXC2) without hydrolysis and for various periods of hydrolysis: 1 month, 2 months, 3 months, 5 months, and 8 months;

FIG. 3 is a table showing the residual mechanical strengths (ball burst values) measured by relating the mechanical strength at T1, 2, 3, 5, or 8 months to the initial mechanical strength T0 multiplied by 100, the mechanical strength being indicated in FIG. 2;

FIG. 4 is a graph showing on the y-axis the residual ball burst values (%), as a function of the duration of hydrolysis on the x-axis for several examples of implantable device according to this disclosure and comparative examples of implantable devices;

FIG. 5 is a graph showing on the y-axis the ball burst values (N/cm), as a function of the duration of hydrolysis on the x-axis for several examples of implantable device according to this disclosure and comparative examples of implantable devices;

FIG. 6 shows the mesh pattern of the first example of implantable device according to this disclosure and comparative examples of implantable device.

### DETAILED DESCRIPTION

This disclosure responds to the above-cited problems in that an object of this disclosure, according to a first aspect, is a method for inducing an improvement in the mechanical strength (N/cm) of an implantable device after a determined period of hydrolysis of the implantable device, in particular measured in weeks or in months, the implantable device comprising one or more yarns comprising at least one lactic acid (co)polymer, the method further comprising the steps advantageously taking place in this order:
i)—a step of providing an implantable device comprising one or more yarns comprising at least one lactic acid (co)polymer;
ii)—a step of treating the implantable device with supercritical carbon dioxide (CO₂);
iii)—a step of obtaining an implantable device having a mechanical strength greater than or equal to 10 N/cm or 15N/cm or 20N/cm or 22N/cm, preferably greater than or equal to 25 N/cm or 27 N/cm or 29N/cm, after 20 weeks of hydrolysis, in particular in an aqueous medium. It was surprisingly discovered that the treatment by scCO₂ of an implantable device, at least partially made of fabric and at least partially resorbable, with yarns made of lactic acid (co)polymer, can improve the mechanical strength of this implantable device during its hydrolysis.

Thus, it was observed that an at least partially textile, implantable device comprising monofilamentary yarns made of lactic acid (co)polymer treated by scCO₂ has a mechanical strength (N/cm) multiplied by three at least at 5 months of hydrolysis compared with an identical implantable device that has not been treated by scCO₂.

### Implantable Device

The implantable device preferably comprises, in particular is, a flat or three-dimensional textile plate or comprises a flat portion and a three-dimensional portion.

In the present document, flat textile plate or flat portion is understood to mean that this portion or this plate extends in a two-dimensional plane, along axes x and y, the thickness of the plate or portion being not significant and/or not considered.

In the present document, three-dimensional textile plate or three-dimensional portion is understood to mean that this portion or this plate extends in three dimensions, along axes x, y and z, the thickness of the plate or of the portion being measured along the z-axis.

Preferably, the implantable device comprises one or more textiles, in particular one or more knitted and/or woven and/or braided textile panels, more preferably knitted.

Preferably, the implantable device, in particular the textile or textile panel, has a mass per unit area greater than or equal to 10 g/m² or 20 g/m² or 30 g/m² or 40 g/m².

More preferably, the implantable device, in particular the textile or textile panel, has a mass per unit area greater than or equal to 50 g/m² or 60 g/m² or 70 g/m² or 80 g/m² or 90 g/m² or 100 g/m², preferably greater than or equal to 110 g/m².

Preferably, the implantable device, in particular the textile or the textile panel, has a mass per unit area less than or equal to 300 g/m² or 250 g/m² or 230 g/m² or 220 g/m² or 200 g/m², more preferably less than or equal to 190 g/m² or 180 g/m².

Advantageously, the flat or three-dimensional textile plate comprises, or substantially consists of, one or more textiles.

In an embodiment, the knitted fabric is weft-knitted or warp-knitted (i.e. a warp-knitted fabric)

Preferably, the implantable device comprises, or the textile or the textile plate is, a warp-knitted fabric, more preferably the warp-knitted fabric comprises one or more monofilamentary yarns.

Preferably, the implantable device is arranged for treating an abdominal hernia, for example a ventral or inguinal hernia.

Advantageously, the implantable device is at least partially textile.

Advantageously, the implantable device and/or the textile and/or the textile plate is/are at least partially resorbable, preferably at least 50% or 60% or 70% or 80% or 90% or 95% or 99% or 100% approximately by mass of the implantable device or of the textile or of the plate is resorbable.

In the present document, resorbable shall mean that all or part of the implantable device and/or of a textile and/or of a textile plate is resorbable by hydrolysis, in other words that it is progressively degraded, chemically and/or mechanically, by hydrolysis in an aqueous medium at ambient temperature (for example at a temperature greater than or equal to 20° C., or at 37° C.) at the end of a determined period and/or once implanted in a living organism (bioresorbable), in particular a mammal, more particularly the human body, at the end of a determined period by hydrolysis, for example 4 months or 5 months or 8 months or 10 months or 12 months or more.

A textile can comprise one or more monofilamentary yarns and/or a plurality of multifilamentary yarns.

Preferably, the aqueous medium comprises at least 50% or 60% or 70% or 80% or 90%, by mass or by volume, of water, in particular distilled and/or osmosis water.

Preferably, the aqueous medium has a pH greater than or equal to 6 and less than or equal to 8, more preferably greater than or equal to 6.5 or 6.7 or 6.8 or 7.0 or 7.2, preferably less than or equal to 7.8 or 7.6.

For example, the aqueous medium has a pH of order 7.4+/−0.1.

Preferably, the aqueous medium is buffered with a phosphate-buffered saline solution (PBS).

Preferably, a monofilamentary yarn has a diameter greater than or equal to 0.01 mm, more preferably greater than or equal to 0.05 mm, most preferably greater than or equal to 0.10 mm.

Preferably, a monofilamentary yarn has a diameter less than or equal to 3 mm, more preferably less than or equal to 2 mm or 1 mm or 0.80 mm, most preferably less than or equal to 0.70 mm or 0.60 mm or 0.50 mm or 0.40 mm or 0.30 mm or 0.20 mm.

Preferably, the one or more monofilamentary yarns each comprise or consist of, one or more (bio)resorbable materials chosen from: one or more L-form or D-form lactic acid polymer, a glycolic acid polymer, a copolymer of lactic acid (L and/or D) and glycolic acid, polycaprolactone, poly-4-hydroxybutyrate (P4HB), or a mixture thereof.

The L (laevorotatory) and D (dextrorotatory) forms are two isomers of lactic acid.

The lactic acid polymer can be a homopolymer or a copolymer.

In the present document, copolymer is understood to mean any polymer comprising at least two different repetition units, and optionally at least three different repetition units (for example a terpolymer).

Lactic acid (co)polymer is understood to mean a lactic acid homopolymer (comprising L-form and/or D-form repetition units), and any copolymer comprising at least lactic acid repetition units (comprising L-form and/or D-form repetition units) and repetition units different from lactic acid, for example glycolic acid.

In an embodiment, the one or more monofilamentary yarns (each) have an elongation at break less than or equal to 100%, in particular less than or equal to 70%, more particularly less than or equal to 60% or 50% or 45%.

Advantageously, the one or more monofilamentary yarns (each) have an elongation at break greater than or equal to 10%, in particular greater than or equal to 15% or 20%.

In an embodiment, the one or more monofilamentary yarns (each) have a tenacity (cN/dtex) greater than or equal to 1 cN/dtex, preferably greater than or equal to 2 or 3 cN/dtex.

Advantageously, the one or more monofilamentary yarns (each) have a tenacity (cN/dtex) less than or equal to 30 cN/dtex, preferably less than equal to 20 cN/dtex, more preferably less than or equal to 15 cN/dtex, in particular less than or equal to 10 cN/dtex.

In an embodiment, the one or more monofilamentary yarns (each) have a breaking load greater than or equal to 300 cN, preferably greater than or equal to 400 cN or 500 cN or 600 cN or 700 cN or 800 cN.

Advantageously, the one or more monofilamentary yarns (each) have a breaking load less than or equal to 3000 cN, preferably less than or equal to 2000 cN or 1500 cN.

The breaking load, tenacity and elongation at break of the monofilamentary yarn have been measured according to standard EN 13895 dated June 2003, and entitled “Textiles—Monofilaments—Determination of tensile properties”.

In an embodiment, the one or more monofilamentary yarns (each) have a linear density (dtex) greater than or equal to 50 dtex, preferably greater than or equal to 100 dtex or 50 dtex or 180 dtex or 200 dtex.

Advantageously, the one or more monofilamentary yarns (each) have a linear density (dtex) less than or equal to 800 dtex, preferably less than or equal to 700 dtex or 600 dtex or 500 dtex or 400 dtex or 300 dtex, for example of order 220 dtex+/−50 dtex.

The linear density is measured using standard NF EN 13392 dated September 2001, entitled “Textiles—Monofilaments—Determination of linear density”.

Monofilamentary yarn is understood to mean any yarn comprising a single filament, and capable of being used on a textile loom, in particular a knitting loom, a weaving loom or a braiding loom.

In an embodiment, the implantable device comprises a warp-knitted fabric comprising monofilamentary yarns, in particular:
a first monofilamentary yarn forms open and/or closed knitted stitches, in particular atlas stitches, more particularly extending over at least three columns of stitches (or over at least three needles), in particular over at most 20 or 15 or 10 columns of stitches (or needles); and
a second monofilamentary form of open and/or closed knitted stitches, in particular atlas stitches, more particularly extending over at least three columns of stitches (or over at least three needles), in particular over at most 20 or 15 or 10 columns of stitches (or needles), in particular the second yarn is supported by a guiding bar working in a direction opposite to the guiding bar supporting the first monofilamentary yarn; and
optionally, a third monofilamentary yarn forms open and/or closed knitted stitches, in particular atlas stitches, more particularly extending over at least three columns of stitches (or over at least three needles), in particular over at most 20 or 15 or 10 columns of stitches (or needles), in particular according to the same pattern of stitches as that of the first monofilamentary yarn; and
optionally a fourth monofilamentary yarn forms open and/or closed knitted stitches, in particular atlas stitches, more particularly extending over at least three columns of stitches (or over at least three needles), in particular over at most 20 or 15 or 10 columns of stitches (or needles), in particular the fourth yarn is supported by a guiding bar travelling in a direction opposite to the guiding bar supporting the third monofilamentary yarn.

In an embodiment, the knitted fabric comprises:
a first monofilamentary yarn threaded, in particular fully, on a first guide bar B1 according to the following described weave, in accordance with standard ISO 11676, dated 2014: 2-3/2-1/2-3/2-1/1-0/1-2/1-0/1-2//.
a second monofilamentary yarn threaded, in particular fully, on a second guide bar B2 according to the following described weave in accordance with standard ISO 11676, dated 2014: 1-0/1-2/1-0/1-2/2-3/2-1/2-3/2-1//;
and optionally:
a third monofilamentary yarn threaded, in particular fully, on a third guide bar B3 according to the following described weave in accordance with standard ISO 11676, dated 2014: 2-3/2-1/2-3/2-1/1-0/1-2/1-0/1-2//; and/or
a fourth monofilamentary yarn threaded, in particular fully, on a fourth guide bar B4 according to the following described weave in accordance with standard ISO 11676, dated 2014: 1-0/1-2/1-0/1-2/2-3/2-1/2-3/2-1//.

Preferably, bar B1 moves/knits in a direction opposite to the direction of movement/knitting of bar B2.

Preferably, bar B3 moves/weaves in a direction opposite to the direction of movement/knitting of bar B4.

Preferably, bar B1, respectively B2, moves/weaves in the same direction as the direction of movement/knitting of bar B3, respectively B4.

Advantageously, working with guide bars having the same movement makes it possible to knit with a single yarn per bar, and thus to better balance the tensions exerted on a yarn than if one bar supports two yarns, while increasing the mass per unit area of the knitted fabric.

For example, a knitted fabric with two bars B1 and B2, each bar supporting one yarn, makes it possible to obtain a mass per unit area of approximately 120 g/m²+/−20 g/m².

For example, a knitted fabric with four bars B1, B2, B3 and B4, each bar supporting one yarn, makes it possible to obtain a mass per unit area of approximately 170 g/m²+/−20 g/m².

The implantable device can comprise a textile, for example a knitted fabric, comprising one or more resorbable polymer coatings, for example based on polyvinylpyrrolidone, a cellulose polymer, a cyclodextrin polymer, a k-carrageenan polymer, or a combination thereof.

### Improvement of the Mechanical Strength (N/Cm)

The mechanical strength (newtons/cm), or bursting value (newtons/cm), is measured according to the test method described in standard ASTM D3787-7 (2011), entitled “Bursting Strength of Textiles—Constant-Rate of Traverse (CRT) Ball Burst Test”

In particular, this standard enables a total breaking strength F to be determined, expressed in newtons. The breaking strength expressed in N/cm is calculated by dividing the total breaking strength expressed in newtons by the perimeter of the ball expressed in cm.

In particular, the diameter of the ball conforming to ASTM D3787-7 (2011), is 2.54 cm (i.e. an “Imperial inch” or an “International inch”) and therefore has a circumference of 2.54×Pi in cm. The breaking strength expressed in N/cm is therefore calculated according to the formula: F/(2.54×Pi).

In particular, the tests are performed on an average of three to six textile samples, in particular six, of approximately 10 cm*10 cm. More particularly, the sample (10 cm×10 cm) is firmly fixed, without tension, between two circular grooved plates. Then, the movable accessory, comprising a rod having an end comprising a ball, is fixed on the traction jaw and the accessory moves towards the surface of the textile sample in order that the ball perforates the textile sample at a constant movement speed of 305 mm/min. A force is exerted against the surface of the textile sample by the steel ball (the diameter of the ball is, in particular, one inch, i.e. 2.54 cm). The bursting value of the ball corresponds to the force necessary to pierce the sample. This value (in newtons) is divided by the circumference of the ball of diameter one imperial inch (2.54×Pi=7.98 cm approximately) in order to obtain the strength value in newtons per centimetre (N/cm).

This test can advantageously use the entire textile surface of the tested sample.

Advantageously, the implantable device, in particular the (flat or three-dimensional) textile plate or the knitted fabric, has a mechanical strength or bursting value before implantation or at 0 days of hydrolysis, greater than or equal to 16 N/cm for the treatment of an inguinal hernia.

Advantageously, the implantable device, in particular the textile plate (i.e. flat or three-dimensional) or the knitted fabric, has a mechanical strength or bursting value before implantation or at 0 days of hydrolysis, greater than or equal to 32 N/cm for the treatment of a ventral hernia.

Preferably, the implantable device, in particular the textile plate (i.e flat or three-dimensional) or knitted fabric, has a mechanical strength (N/cm) of R0 (N/cm) before implantation or at 0 days of hydrolysis, and a mechanical strength (N/cm) R1 at the end of 3 months of hydrolysis, with R1 less than or equal to 0.98×R0 or 0.93×R0 and greater than or equal to 0.60×R0 or 0.70 or 0.80×R0.

Preferably, the implantable device has a mechanical strength (N/cm) of R0 (N/cm) before implantation or at 0 days of hydrolysis, and a mechanical strength (N/cm) R2 at the end of 5 months hydrolysis, with R2 less than or equal to 0.94×R0 or 0.89×R0 and greater than or equal to 0.50×R0 or 0.60×R0.

Preferably, the implantable device has a mechanical strength (N/cm) of R0 (N/cm) before implantation or at 0 days of hydrolysis, and a mechanical strength (N/cm) R3 at 8 months of hydrolysis, with R3 less than or equal to 0.60 or 0.50×R0 and greater than or equal to 0.20 or 0.30 or 0.35×R0. Preferably, the reduction in mechanical strength observed for the implantable device treated according to this disclosure is between a minimum of 10% and a maximum of 27% at the end of 3 months of hydrolysis compared with more than 30% for an implantable device of identical structure but not treated according to this disclosure by scCO₂.

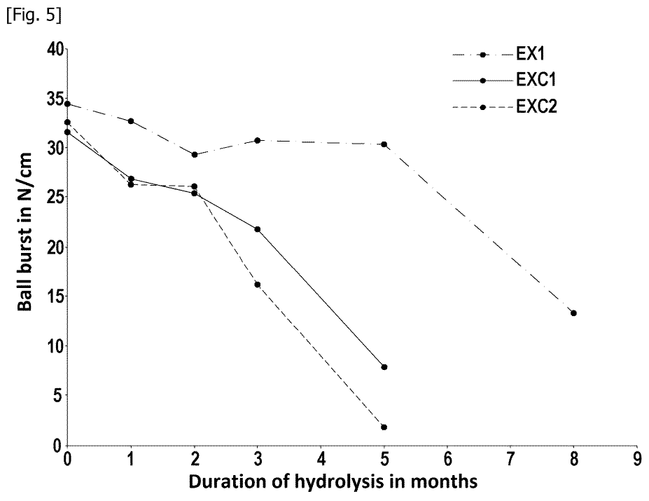

Advantageously, the reduction in mechanical strength (N/cm) observed for the implantable device treated according to this disclosure with scCO₂, in particular deduced from the curve EX1 shown in attached FIG. 4, is approximately 12% at the end of 5 months of hydrolysis and 61% at the end of 8 months of hydrolysis, compared with 95% at the end of 5 months of hydrolysis and 100% at the end of 8 months of hydrolysis, in particular deduced from curve EXC2 shown in attached FIG. 4, for an implantable device of identical structure but not treated according to this disclosure by scCO₂.

scCO₂, or Supercritical Carbon Dioxide or Carbon Dioxide in a Supercritical State

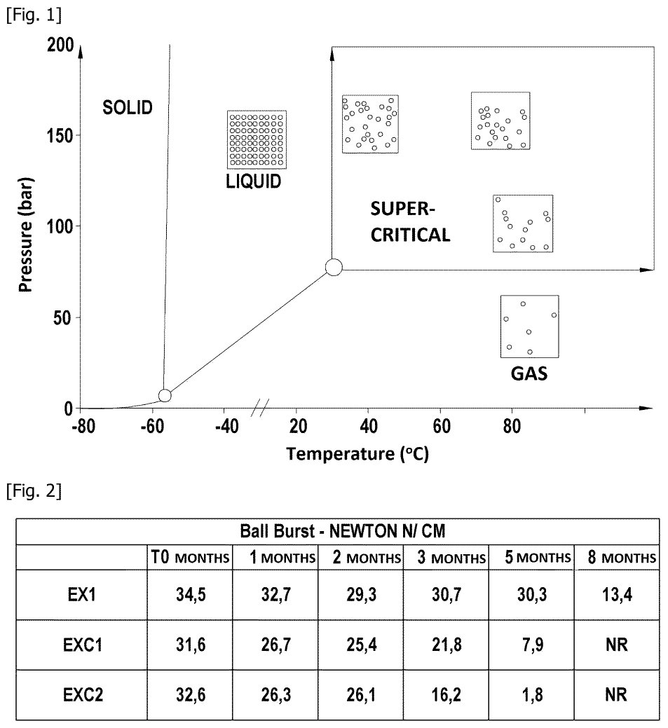

Pure carbon dioxide enters a supercritical state when it is subjected to a pressure greater than a critical pressure and heated to a temperature above a critical temperature, in particular according to the diagram of pressure (bar) and temperature (° C.) shown in attached FIG. 1.

Advantageously, the critical pressure is greater than or equal to 73 bar, and the critical temperature is greater than or equal to 31° C.

Advantageously, carbon dioxide in a supercritical state has particular properties: comparable to those of a liquid for which the density is greater than or equal to 0.2 g/cm³ and less than or equal to 1 g/cm³ with a solvent ability, and comparable to those of a gas with a high diffusivity greater than or equal to 104 cm²/second, and less than or equal to 10⁻³ cm²/second, in particular is miscible with other gases, and/or has a low surface tension.

Advantageously, carbon dioxide in a supercritical state (designated above and below as scCO₂) has a viscosity greater than or equal to 10 μPa·s and less than or equal to 100 μPa·s.

In an embodiment, the treatment step (ii) comprises the following steps, in particular taking place in the following order:
iia) providing liquid carbon dioxide;
iib) applying temperature and pressure conditions to the carbon dioxide enabling the carbon dioxide to pass from a liquid state to a supercritical state;
iic) supplying the carbon dioxide, in a supercritical state to an enclosure, in particular an autoclave, comprising the implantable device, in particular the enclosure comprises a hermetic treatment volume in which the temperature and pressure conditions are determined so as to maintain the carbon dioxide in a supercritical state.

Advantageously, the carbon dioxide is stored in a container in the liquid state during step iia).

Advantageously, step iib) comprises firstly pumping the carbon dioxide in the liquid state and applying a determined pressure in order to transform the carbon dioxide in the liquid state into a gaseous state, then the carbon dioxide in the gaseous state is heated to a determined temperature in order to transform the carbon dioxide in the gaseous state into carbon dioxide in a supercritical state.

Advantageously, step iic) comprises pumping and supplying the treatment volume of the enclosure with scCO₂.

Advantageously, after step iic), the pressure of the scCO₂ is reduced, for example to a pressure determined on the diagram of FIG. 1, in order to obtain a gaseous state, then the gaseous CO₂ obtained undergoes a separation step for separating the CO₂ from any contaminants, the resulting gaseous CO₂ is condensed in order to attain a liquid state, and optionally supplied to the storage container (for example to undergo a new treatment cycle according to step ii)).

Preferably, in step ii), in particular in step iib) and/or iic), the carbon dioxide is heated, in particular held in step iic), at a temperature greater than or equal to 10° C., more preferably greater than or equal to 20° C., most preferably greater than or equal to 25° C. or 30° C. or 33° C. or 35° C.

Preferably, in step ii), in particular in step iib) and/or iic), the carbon dioxide is heated, in particular held in step iic), at a temperature less than or equal to 100° C., more preferably less than or equal to 80° C., most preferably less than or equal to 70° C. or 60° C. or 55° C. or 50° C.

Preferably, in step ii), in particular in step iib) and/or iic), the carbon dioxide is placed under pressure, in particular held in step iic), at a pressure greater than or equal to 50 bar, more preferably greater than or equal to 60 bar, yet more preferably greater than or equal to 70 bar or 80 bar or 90 bar, most preferably greater than or equal to 100 bar or 110 bar or 120 bar or 130 bar or 140 bar or 150 bar.

Preferably, in step ii), in particular in step iib) and/or iic), the carbon dioxide is placed under pressure, in particular held in step iic), at a pressure less than or equal to 600 bar, more preferably less than or equal to 500 bar, yet more preferably less than or equal to 480 bar or 460 bar or 440 bar, most preferably less than or equal to 420 bar or 380 bar or 360 bar or 340 bar or 320 bar or 300 bar or 250 bar or 200 bar.

In an embodiment, in step ii), in particular in step iib) and/or iic), the carbon dioxide is placed under pressure at a pressure between 100 bar and 300 bar and heated to a temperature between 35° C. and 50° C.

In a preferred embodiment, in step ii), in particular in step iib) and/or iic), the carbon dioxide is placed under pressure at a pressure of approximately 150 bar+/−20 bar, and heated to a temperature of approximately 38° C.+/−5° C.

Preferably, the treatment period for the implantable device in step ii), in particular in step iic) is greater than or equal to 5 minutes, more preferably greater than or equal to 10 minutes or 15 minutes or 20 minutes, most preferably greater than or equal to 25 minutes or 30 minutes.

Preferably, the treatment period for the implantable device in step ii), in particular in step iic), is less than or equal to 60 minutes, more preferably less than or equal to 50 minutes or 40 minutes or 35 minutes.

In a preferred embodiment, the treatment period of the implantable device in step ii), in particular in step iic), is approximately 30 minutes+/−5 minutes.

In a preferred embodiment, the quantity of carbon dioxide in the supercritical state supplied to the enclosure in step iic) is between 500 kg/m³ and 1200 kg/m³, preferably between 600 kg/m³ and 1100 kg/m³, more preferably between 700 kg/m³ and 900 kg/m³, most preferably approximately 800 kg/m³+/−50 kg/m³.

In an embodiment, the volume of scCO₂ supplied in the treatment enclosure corresponds to at least 20%, or at least 30% or at least 40%, preferably at least 50%, of the total treatment volume of the treatment enclosure, more preferably approximately 66%+/−10% of the total treatment volume of the treatment enclosure.

### Hydrolysis Protocol

The hydrolysis of a textile, and in particular of the implantable device according to this disclosure or comparative, comprises the following steps:
one or more samples of the implantable device, in particular one or more textile samples, (each) having dimensions of 10 cm×10 cm is/are introduced into a glass container comprising 10 litres of purified water buffered with a phosphate-buffered saline solution (PBS) in order to obtain a pH of 7.4. The container is made of an inert material, in particular glass, and hermetically sealed with a cover, thus avoiding loss of water by evaporation (the volume of 10 litres therefore remains constant). This container is introduced into an oven at a temperature of 37° C. Manual stirring is carried out every day for approximately 15 seconds in order to homogenise the hydrolysis medium.

In particular, between each manual stirring, no additional mechanical stirring is carried out.

The sample for which it is desired to analyse the mechanical strength after a period of n months, or n*4 weeks, of hydrolysis is extracted from the container, when dried, in particular in an oven at 37° C. for 60 minutes. The duration of hydrolysis thus corresponds to the time during which the sample is left in the volume of PBS and disposed in the oven at 37° C.

In the present document, n months is understood to mean a duration equivalent to n*4 weeks, n being an integer.

In an alternative embodiment, at least 80% by mass, preferably at least 85% or at least 87% or at least 89% or at least 91% or at least 93% or at least 95% or at least 97% or at least 99% or approximately 100% by mass, of the total mass of the implantable device provided in step i) is resorbable, in particular bioresorbable.

Preferably, at least 85% or at least 87% or at least 89% or at least 91% or at least 93% or at least 95% or at least 97% or at least 99% or approximately 100% by mass, of the total mass of the implantable device and/or of a textile (which comprises the implantable device) provided in step i) is formed of one or more (co)polymers chosen from:
an L-form, D-form or L-form and D-form lactic acid polymer, one or more glycolic acid polymers, one or more (L and/or D) lactic acid and glycolic acid copolymers, or a mixture thereof, preferably of one or more L-form, D-form or L-form and D-form lactic acid polymers (list I); and/or
is formed of one or more monofilaments each comprising one or more (co)polymers chosen in the list I above.

In an alternative embodiment, the method comprises a step iv) of sterilising the implantable device performed after step iii), in particular comprising the application of ethylene oxide to the implantable device. Advantageously, any sterilisation method known to a person skilled in the art can be used.

In an alternative embodiment, the implantable device of step iii) has a mechanical strength, or bursting value (newtons/cm), greater than or equal to 10 N/cm after 32 weeks or 8 months of hydrolysis, in particular in an aqueous medium buffered with a phosphate saline solution at a pH of 7.4 and held at a temperature of 37° C. in a hermetically sealed medium.

In an alternative embodiment, the temperature of the supercritical CO₂ during step ii), in particular during step iib) and/or iic), is less than or equal to 70° C., in particular greater than or equal to 10° C.

In an alternative embodiment, the treatment step ii), in particular during step iib) and/or iic), comprises the application of supercritical CO₂ at a pressure greater than or equal to 50 bar, in particular less than or equal to 400 bar.

Advantageously, the applied temperature and pressure do not change the mechanical properties of the implantable device.

In an alternative embodiment, the treatment step ii), in particular during step iib) and/or step iic), comprises the application of supercritical CO₂ at a pressure greater than or equal to 100 bar.

In an alternative embodiment, the implantable device provided in step i) comprises one or more monofilamentary yarns each comprising at least one lactic acid (co)polymer, in particular an L-form or D-form lactic acid polymer, or a combination thereof.

In an alternative embodiment, the one or more monofilamentary yarns each comprising at least one lactic acid (co)polymer, each have a diameter greater than or equal to 50 μm (i.e. 0.050 mm) and less than or equal to 500 μm (i.e. 0.50 mm), in particular less than or equal to 300 μm (i.e. 0.30 mm).

In an alternative embodiment, the at least one lactic acid (co)polymer has a glass transition temperature (Tg) greater than or equal to 40° C. and less than or equal to 150° C.

Preferably, the at least one lactic acid (co)polymer has a glass transition temperature (Tg) greater than or equal to 50° C., more preferably greater than or equal to 60° C., yet more preferably greater than or equal to 70° C., most preferably greater than or equal to 80° C.

Preferably, the at least one lactic acid (co)polymer has a glass transition temperature (Tg) less than or equal to 120° C., more preferably less than or equal to 110° C., yet more preferably less than or equal to 100° C., most preferably less than or equal to 95° C.

In an alternative embodiment, the at least one lactic acid (co)polymer has a melting point (Tf) greater than or equal to 130° C. and less than or equal to 210° C.

Preferably, the at least one lactic acid (co)polymer has a melting point (Tf) greater than or equal to 140° C., more preferably greater than or equal to 150° C., yet more preferably greater than or equal to 160° C., most preferably greater than or equal to 165° C.

Preferably, the at least one lactic acid (co)polymer has a melting point (Tf) less than or equal to 200° C., more preferably less than or equal to 190° C., most preferably less than or equal to 180° C.

Preferably, the at least one lactic acid (co)polymer has a degree of crystallinity greater than or equal to 30%, or 40% or 45%, more preferably greater than or equal to 50% or 53%.

Preferably, the at least one lactic acid (co)polymer has a degree of crystallinity less than or equal to 80%, or 70% or 65%, more preferably less than or equal to 60%.

Advantageously, degree of crystallinity shall mean the ratio of the total sum by mass or by volume of the crystalline fractions of a given sample, relative to the total mass or volume of the sample.

Advantageously, the glass transition temperature, the melting point, and the degree of crystallinity, are determined on the monofilamentary yarn comprising the lactic acid (co)polymer.

Preferably, the melting point and glass transition temperature, and the degree of crystallinity, are measured using the following standards: ISO 11357-2 (2013) entitled “Determination of the glass transition temperature and step height”, ISO 11357-3 (2018) entitled “Determination of the temperature and enthalpy of melting and crystallisation”.

The apparatus is a power-compensated DSC, DSC Q2000 (TA Instruments). The operating conditions are preferably as follows: the method of the apparatus is standard, the sample crucibles are made of hermetic aluminium, the sweep gas is U-grade nitrogen (50 ml/min), the temperature ramp is isothermal at 25° C. for 5 minutes, then increases from 25° C. to 250° C. with a gradient of 10° C. per minute. The test tubes are held for two hours at ambient temperature (23° C.) and approximately 50%+/−10% relative humidity.

In an alternative embodiment, the at least one lactic acid (co)polymer has a mass average molar mass Mw greater than or equal to 70,000 g/mole and less than or equal to 300,000 g/mole.

Preferably, the at least one lactic acid (co)polymer has a mass average molar mass Mw greater than or equal to 80,000 g/mole, or 90,000 g/mole, or 100,000 g/mole, more preferably greater than or equal to 110,000 g/mole, or 120,000 g/mole, or 130,000 g/mole, or 140,000 g/mole, or 145,000 g/mole.

Preferably, the at least one lactic acid (co)polymer has a mass average molar mass Mw less than or equal to 250,000 g/mole, or 230,000 g/mole, or 210,000 g/mole, more preferably less than or equal to 200,000 g/mole, or 190,000 g/mole, or 180,000 g/mole, or 170,000 g/mole, or 160,000 g/mole.

In an alternative embodiment, the at least one lactic acid (co)polymer has a number average molar mass Mn greater than or equal to 10,000 g/mole and less than or equal to 120,000 g/mole or 110,000 g/mole.

Preferably, the at least one lactic acid (co)polymer has a number average molar mass Mn greater than or equal to 20,000 g/mole, or 30,000 g/mole, more preferably greater than or equal to 40,000 g/mole, or 45,000 g/mole.

Preferably, the at least one lactic acid (co)polymer has a number average molar mass Mn less than or equal to 90,000 g/mole or 80,000 g/mole, more preferably less than or equal to 70,000 g/mole or 60,000 g/mole.

In an alternative embodiment, the at least one lactic acid (co)polymer has a polydispersity Ip (Mw/Mn) greater than or equal to 1.5 or 2 and less than or equal to 5, preferably less than or equal to 4, more preferably less than or equal to 3.5 or 3.

Advantageously, the weight and number average molar masses (g/mole) are determined on the monofilamentary yarn comprising the lactic acid (co)polymer.

Preferably, the number and weight average molar masses are measured using standard ASTM D3536-91 entitled “Standard Test Method for Molecular Weight Averages and Molecular Weight Distribution by Liquid Exclusion Chromatography (Gel Permeation Chromatography—GPC)”. This technique is also called stearic exclusion chromatography.

The operating conditions are preferably as follows: Agilent 1260 Infinity GPC systems, Waters Styragel 4-column system, 300*4.6 mm, 5 μm particles, porosity 50 at 104 A, thermostatted assembly at 40° C., analysis-grade THE eluent filtered with a flow rate of 0.5 ml/min, prefiltration of samples on a 0.2 μm PTFE filter, dual IR and UV detection (254 nm), polystyrene calibration, sample preparation by dissolution in a tetrahydrofuran (THF)/Toluene mixture (used as a marker).

In an alternative embodiment, the at least one lactic acid (co)polymer comprises L-form lactic acid units and D-form lactic acid units.

Advantageously, the L and D forms as well as their proportions are determined on the monofilamentary yarn comprising the lactic acid (co)polymer.

Preferably, in order to determine the optical rotation, the samples to be tested are dissolved in chloroform at a concentration of 1 g/dl.

Preferably, the D-form or L-form monomer is assayed by gas phase chromatography coupled with mass spectrometry.

The operating conditions are preferably as follows: each sample is extracted in dichloromethane, the polymer is precipitated in hexane, and the mixture is filtered on a 0.2 μm PTFE syringe filter, the assay is carried out by external calibration (D- or L-form lactide monomer) with the help of four standards prepared at different concentrations; the chromatography conditions are preferably as follows: Agilent HP5975C spectrometer, Aiglent GC 7890, HP-5MS Column (5% polyphenylsiloxane) 30 m, 0.25 μm thickness, 0.25 mm diameter, isotherm for 1 minute at 50° C. then heating at 25° C./min from 50° C. to 320° C., isotherm for 5 minutes at 320° C., the injector is heated to 200° C., 2 μl injection in splitless mode, detection in SCAN mode.

In an alternative embodiment, the at least one lactic acid (co)polymer comprises at least 80% by mass L-form lactic acid units and less than 20% by mass D-form lactic acid units.

Preferably, the at least one lactic acid (co)polymer comprises at least 85% or 88% or 90% or 92% or 94% or 96% or 98% by mass L-form lactic acid units.

Preferably, the at least one lactic acid (co)polymer comprises at most 15% or at most 10% or at most 8% or at most 6% by mass D-form lactic acid units, more preferably at most 5% or 4% by mass D-form lactic acid units.

Preferably, the at least one lactic acid (co)polymer comprises at least 0.5% or 1% or 1.5% by mass D-form lactic acid units.

In an alternative embodiment, at least 80%, preferably at least 85% or 90% or 95%, by mass of the implantable device comprises one or more monofilamentary yarns in one or more lactic acid (co)polymers.

The improvement in the mechanical resistance to hydrolysis, or bursting value, is significant after 3 months of hydrolysis for an implantable device comprising warp-knitted monofilamentary yarns of lactic acid (co)polymer, and in particular with a lactic acid polymer as described above.

In an alternative embodiment, the method comprises a thermosetting step of the one or more textiles (that comprises the implantable device) or of the implantable device, preferably taking place before step ii) or after step ii).

Preferably, the thermosetting step comprises the application of a temperature greater than or equal to 80° C. and less than or equal to 130° C., more preferably greater than or equal to 90° C. and less than or equal to 120° C., most preferably 110° C.+/−5° C.

Preferably, the duration of the thermosetting step is greater than or equal to 30 seconds, more preferably greater than or equal to 60 seconds, most preferably of approximately 120 seconds+/−30 seconds.

Preferably, the duration of the thermosetting step is less than or equal to 2 hours, more preferably less than or equal to 1 hour, most preferably less than or equal to 30 minutes or 20 minutes or 10 minutes or 5 minutes.

Preferably, no tensile force is exerted on the one or more knitted fabrics or the implantable device during the thermosetting step, in particular the one or more knitted fabrics or the devices are free from tension.

An object of the present disclosure according to a second aspect, is an implantable device, in particular for the treatment of an abdominal hernia, in particular a ventral or inguinal hernia, that can be obtained by the method according to any one of the alternative embodiments with reference to the first aspect of this disclosure and/or as described in the present document.

Advantageously, the implantable device obtained has an improved mechanical strength or bursting value at the end of three to eight months of hydrolysis compared with a similar device that has not being treated by supercritical scCO₂.

In an alternative embodiment, at least 80% by mass, preferably at least 85% or 90% or 95% or 98% by mass, of the implantable device is formed of one or more monofilamentary yarns in one or more lactic acid (co)polymers, and the implantable device has a mechanical strength greater than or equal to 10 N/cm, preferably greater than or equal to 15 N/cm or 20 N/cm, more preferably 25 N/cm, at the end of 20 weeks of hydrolysis.

The alternatives and definitions according to the first aspect of this disclosure apply independently of one another to the implantable device according to a second aspect of this disclosure.

An object of the present disclosure according to a third aspect, is the use of supercritical CO₂ for improving the mechanical strength (N/cm), or bursting value (N/cm), of an implantable device comprising one or more yarns comprising at least one lactic acid (co)polymer at the end of a determined period of hydrolysis, in particular in a determined aqueous medium, in particular at the end of a period of at least 20 weeks in order to obtain a mechanical strength greater than or equal to 10 N/cm, preferably greater than or equal to 15 N/cm or 20 N/cm, more preferably greater than or equal to 25 N/cm.

In particular, the use comprises the application of supercritical CO₂ to the implantable device and/or to the monofilamentary yarns.

The alternatives and definitions according to the first aspect of this disclosure and/or the second aspect of this disclosure apply independently from one another to the third aspect of this disclosure.

The present text also comprises, according to a fourth aspect, a method for using an implantable device comprising:
implementing the method for inducing an improvement in the mechanical strength of an implantable device comprising one or more yarns, each comprising one or more lactic acid (co)polymers according to any one of the alternative embodiments with reference to the first aspect of this disclosure, or providing an implantable device according to a second aspect of this disclosure or that can be obtained by the method for inducing an improvement in the mechanical strength according to any one of the alternative embodiments with reference to the first aspect of this disclosure, and
the selection of a group of patients chosen from the following groups: patients having a BMI greater than or equal to 30, patients suffering from type I and/or II diabetes, patients who are smokers (or tobacco users), and patients chosen from a combination of the groups of patients;
the treatment of the patient group selected for treatment of an abdominal hernia, in particular ventral or inguinal hernia, with the implantable device. BMI is the acronym for “Body Mass Index” calculated by dividing the weight (kg) by the height (cm) squared.

FIG. 1 shows the various states: solid, liquid, gas and supercritical of carbon dioxide depending on the conditions of pressure (bar) and temperature that are applied to it.

Thus, a so-called triple point is observed at −56° C. and 5.18 bar at the intersection of which the boundaries are observed between solid, liquid and gaseous state.

The critical point above which carbon dioxide is in a supercritical state comprises a temperature greater than or equal to 31° C. combined with a pressure greater than or equal to 73.85 bar. It can be seen that in this supercritical state, carbon dioxide is in a condition that is both close to the liquid state, which allows it to behave like a solvent, under conditions between a gas and a liquid which enables it to impregnate the implantable device and under conditions close to those of the gaseous state, which enables it to defuse into the structure of the lactic acid-based monofilamentary yarns. The applicant has surprisingly observed that a treatment by scCO₂, in particular of the textile structure of the implantable device, can significantly improve the mechanical strength of the implantable device during its hydrolysis.

Advantageously, the treatment by scCO₂ according to this disclosure can improve the mechanical behaviour of the implantable device to hydrolysis, in particular reduce the rate of hydrolysis, but it is not a treatment with a view to its sterilisation.

In an embodiment, a sterilisation step, distinct from the step of treatment by scCO₂ for inducing an improvement in the mechanical strength, is necessary. This sterilisation step can be performed by any sterilisation method known in the field of sterilising an implantable device, in particular already disposed in a double bag.

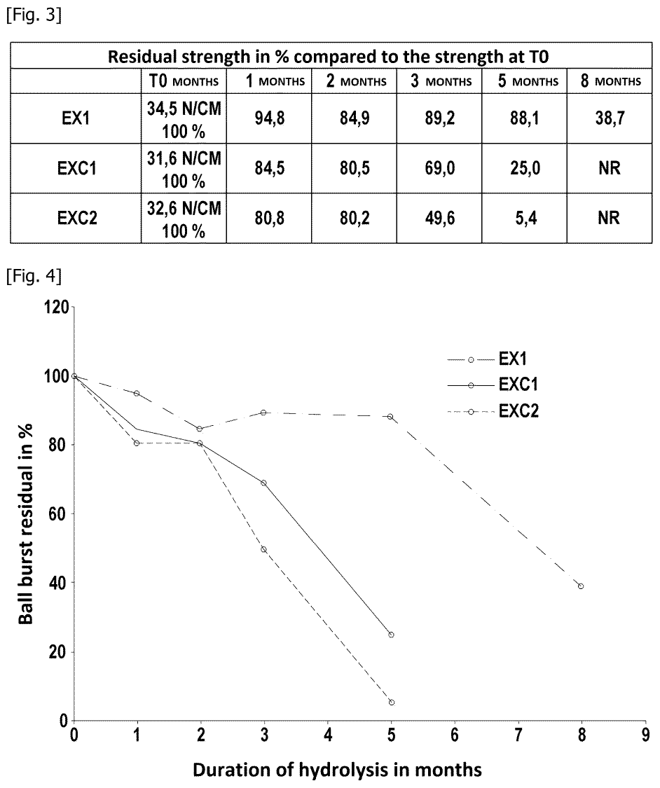

FIGS. 2 to 5 show the results of hydrolysis tests obtained for an exemplary embodiment EX1 according to this disclosure, and two comparative examples EXC1 and EXC2.

Example EX1 is a knitted fabric 10 that is warp-knitted according to the mesh pattern shown in FIG. 6. This knitted fabric comprises a first monofilamentary yarn 20 made of a lactic acid polymer having a diameter of 0.15 mm and a linear density of 220 dtex, and a second monofilamentary yarn 30, similar to the first monofilamentary yarn (made of a lactic acid polymer having a diameter of 0.15 mm and a linear density of 220 dtex). The first yarn 20 is supported by a first guiding bar B1 on the knitting loom, the movement of which is as follows: 2-3/2-1/2-3/2-1/1-0/1-2/1-0/1-2//.

The second yarn 30 is supported by a second guiding bar B2 on the knitting loom, the movement of which is as follows: 1-0/1-2/1-0/1-2/2-3/2-1/2-3/2-1//.

The knitted fabric 10 has a mass per unit area of approximately 120 g/m², and is entirely resorbable.

The first and second guidance bars, or so called guide bars, B1, B2 work/knit over the entire width of the knitted fabric 10 and over its entire height.

Advantageously, the first and seconds guiding bars B1, B2 work in opposition: the first guiding bar supporting yarn 20 works in direction F2 while the second guiding bar supporting yarn 30 works in direction F1, the directions F1 and F2 being opposite as shown in FIG. 6.

In this specific example, the knitted fabric 10 undergoes a thermosetting step at 110° C. for 2 minutes, then undergoes an scCO₂ treatment consisting of placing it in an autoclave at 38° C., under a pressure of 150 bar for 30 minutes in order to keep the CO₂ in its supercritical state. The volume of scCO₂ supplied in the treatment enclosure represents approximately 66% of the total treatment volume of the enclosure.

Comparative examples EXC1 and EXC2 are each of a knitted fabric identical to knitted fabric 10 but not undergoing any scCO₂ treatment. EXC2 undergoes a thermosetting step at 110° C. for 2 minutes similar to the thermosetting step of EX1 while EXC1 does not undergo a thermosetting step.

For the performance of the hydrolysis tests: 30 samples of knitted fabric 10 cm*10 cm are introduced into a stainless steel container containing water buffered with PBS of each example (EX1, EXC1, EXC2) according to the hydrolysis protocol described above. At the end of each targeted period of hydrolysis (1 month, 2 months, 3 months, 5 months, 8 months), 18 samples (6 samples of each example) are removed from the stainless steel container and then dried in an oven at 37° C. for one hour. These 18 samples are all evaluated with the bursting value test described above (ASTM D3787-7 (2011)) immediately after drying.

The mechanical strength evaluated here is advantageously a ball burst value. It could be another value of mechanical strength, such as the breaking load in the warp or weft direction of the knitted fabric, i.e. any measurement allowing an evaluation of the improvement in mechanical strength of the knitted fabric.

The melting point and the degree of crystallinity measured for EX1 at T0 are respectively 169.53° C. and 59%, and at T1 (months) are 167.88° C. and 46%.

The melting point and the degree of crystallinity measured for EXC1 at T0 are respectively 166.76° C. and 56%, and at T1 (months) are 166.83° C. and 56%.

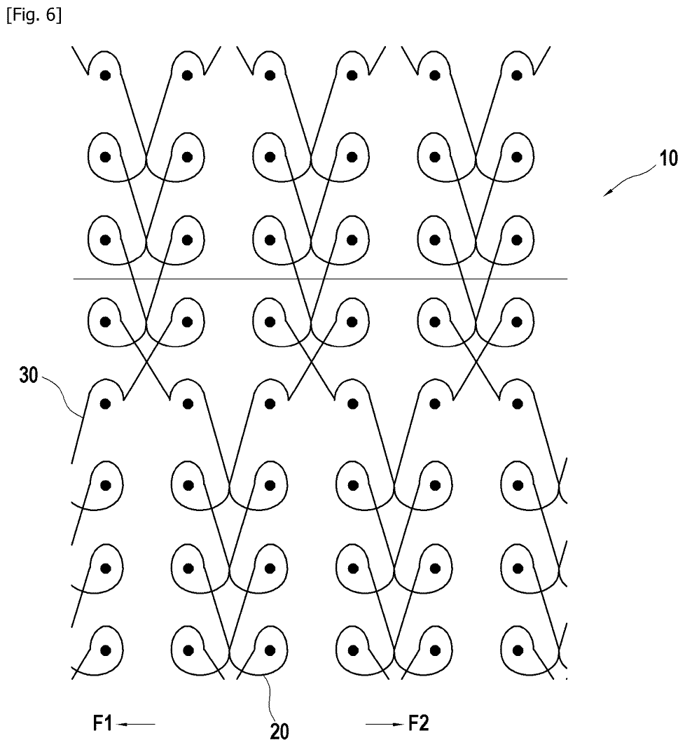

Surprisingly, it can be seen in FIGS. 4 and 5 that already at the end of 2 months of hydrolysis, the mechanical strength of EX1 according to this disclosure is degraded but remains at more than 80% of the initial mechanical strength, while that of examples EXC1 and EXC2 starts to decreased significantly. At the end of three months and up to approximately 5 months, the mechanical strength is still at least more than 80% of the initial mechanical strength for EX1, whereas it drops by more than 40% at three months and more than 75% at 5 months of the initial strength for examples EXC1 and EXC2. Finally, at the end of 8 months of hydrolysis, the knitted fabric according to EX1 still has a mechanical strength corresponding to approximately 40% of its initial strength, whereas the knitted fabrics of examples EXC1 and EXC2 are totally hydrolysed or too hydrolysed for it to be possible to measure a strength value.

The use of scCO₂ to improve the mechanical strength of a textile comprising monofilaments of a lactic acid (co)polymer makes it possible to improve the hydrolysis behaviour thereof, in particular to reduce its rate of hydrolysis, in a simple and reliable manner, from a reliable resorbable textile structure in the treatment of a hernia for example. This provision will thus make it possible to prolong the mechanical strength provided by the implantable device, while limiting the risk of infection, and will avoid resorting to surgery to remove the implantable device in the event of infection, since the device will eventually be completely resorbed.

1. A method for inducing an improvement in the mechanical strength (N/cm) of an implantable device after a determined period of hydrolysis of the implantable device, the implantable device comprising one or more yarns comprising at least one lactic acid (co)polymer, the method comprising:
i)—a step of providing an implantable device comprising one or more yarns comprising at least one lactic acid (co)polymer;
ii)—a step of treating the implantable device with supercritical carbon dioxide (CO₂);
iii)—a step of obtaining an implantable device having a mechanical strength greater than or equal to 10 N/cmafter 20 weeks of hydrolysis.

2. The method according to claim 1, wherein at least 80% by mass of the total mass of the implantable device provided in step i) is resorbable.

3. The method according to claim 1, wherein the method comprises a step iv) of sterilisation of the implantable device performed after step iii).

4. The method according to claim 1, wherein the implantable device of step iii) has a mechanical strength greater than or equal to 10 N/cm after 32 weeks of hydrolysis in an aqueous medium.

5. The method according to claim 1, wherein the temperature of the supercritical CO₂ during step ii) is less than or equal to 70° C.

6. The method according to claim 1, wherein the treatment step ii) comprises the application of supercritical CO₂ at a pressure greater than or equal to 10 bar and less than or equal to 500 bar.

7. The method according to claim 1, wherein the treatment step ii) comprises the application of supercritical CO₂ at a pressure greater than or equal to 100 bar.

8. The method according to claim 1, wherein the implantable device provided in step i) comprises one or more monofilamentary yarns each comprising the at least one lactic acid (co)polymer.

9. The method according to claim 8, wherein monofilamentary yarn(s) each comprising the at least one lactic acid (co)polymer has, or each have, a diameter greater than or equal to 50 μm and less than or equal to 500 μm.

10. The method according to claim 1, wherein the at least one lactic acid (co)polymer has a glass transition temperature (Tg) greater than or equal to 40° C. and less than or equal to 150° C.

11. The method according to claim 1, wherein the at least one lactic acid (co)polymer has a melting point (Tf) greater than or equal to 130° C. and less than or equal to 210° C.

12. The method according to claim 1, wherein the at least one lactic acid (co)polymer has a mass average molar mass Mw greater than or equal to 70,000 g/mole and less than or equal to 300,000 g/mole.

13. The method according to claim 1, wherein the at least one lactic acid (co)polymer has a polydispersity Ip (Mw/Mn) greater than or equal to 1.5 and less than or equal to 5.

14. The method according to claim 1, wherein the at least one lactic acid (co)polymer comprises L-form lactic acid units and D-form lactic acid units.

15. The method according to claim 1, wherein the at least one lactic acid (co)polymer comprises at least 80% by mass L-form lactic acid units and less than 20% by mass D-form lactic acid units.

16. The method according to claim 1, wherein at least 80% by mass of the implantable device comprises one or more monofilamentary yarns in one or more lactic acid (co)polymers.

17. An implantable device for the treatment of an abdominal hernia, which can be obtained by the method according to claim 1.

18. The implantable device according to claim 17, wherein it comprises at least 80% by mass of one or more monofilamentary yarns in one or more lactic acid (co)polymers, and wherein the implantable device has a mechanical strength greater than or equal to 25 N/cm at the end of 20 weeks of hydrolysis.

19. Method for inducing an improvement in the mechanical strength (N/cm) of an implantable device after a determined period of hydrolysis of the implantable device, the implantable device comprising one or more yarns comprising at least one lactic acid (co)polymer, the method comprising using supercritical CO₂.

20. The method according to claim 1, wherein the method comprises:
the selection of a group of patients chosen from the following groups: patients having a Body Mass Index (BMI) greater than or equal to 30, patients suffering from type I diabetes, patients suffering from type II diabetes, patients who are smokers (or tobacco users), and patients chosen from a combination of the groups of patients; and
the treatment of the patient group selected for treatment of an abdominal hernia with the implantable device obtained in step iii).
